(19) United States
(12) Patent Application Publication
(10) Pub. No.: US 2012/0041083 A1
(43) Pub. Date: Feb. 16, 2012

(54) SILICON OR ELEMENTARY METALS AS ENERGY CARRIERS

(75) Inventor: Peter Grauer, Meggen (CH)

(73) Assignee: SILICON FIRE AG, Meggen (CH)

(21) Appl. No.: 13/140,822

(22) PCT Filed: Nov. 13, 2009
(86) PCT No.: PCT/EP2009/065165

§ 371 Date: Sep. 29, 2011

(30) Foreign Application Priority Data
Dec. 18, 2008 (EP) PCT/EP2008/067895
Feb. 6, 2009 (EP) 09152292.0
Sep. 9, 2009 (EP) PCT/EP2009/061707

(51) Int. Cl. C07C 27/00; C07C 29/00

(52) U.S. Cl. 518/702; 568/840

(57) ABSTRACT

A method for providing storable and transportable energy carriers (103, 104) is described. In one step, a transformation of silicon-dioxide-containing or metal-oxide-containing starting material (101) to silicon (103) or a metal occurs in a reduction process (105), wherein the primary energy for this reduction process (105) is provided from a renewable energy source. Then, a portion of the reaction products (102) from the reduction process (105) is applied in a process (106) for generating methanol, wherein (106) a synthesis gas (110) composed of carbon monoxide and hydrogen is used.

### CROSS-REFERENCES TO RELATED APPLICATIONS

The present application claims the priorities of Patent Cooperation Treaty Application No. PCT/EP2009/065165, filed on Nov. 13, 2009; Patent Cooperation Treaty Application No. PCT/EP2009/061707, filed on Sep. 9, 2009; European Patent Application No. 09152292.0, filed Feb. 6, 2009; and Patent Cooperation Treaty Application No. PCT/EP2008/067895, filed Dec. 18, 2008; all of which are incorporated herein by reference in their entirety for all purposes.

### BACKGROUND OF THE INVENTION

The present application relates to methods for providing storable and transportable energy carriers.

Carbon dioxide (often called carbonic acid gas) is a chemical compound of carbon and oxygen. Carbonic acid gas is a color- and odorless gas. It is a natural, component of the air with a little concentration and is generated in animals (resp. living beings) in the cell respiration, but also in the combustion of carbon-containing substances under (supply of) sufficient oxygen. Since the advent of the industrialization, the CO₂ proportion in the atmosphere has risen significantly. A main cause for this are the CO₂ emissions caused by humans, the so-called anthropogenic CO₂ emissions. The carbonic acid gas in the atmosphere absorbs a portion of the heat radiation. This property renders carbonic acid gas to be a so-called green-house gas and is one of the co-originators of the green-house effect.

For these and also for other reasons, research and development is performed at present to find a way to reduce the anthropogenic CO₂ emissions. In particular, for the generation of power, which is often carried out by the combustion of fossil energy carriers, such as coal or gas, but also in other combustion processes, for example in waste incineration, there is a strong demand for reducing CO₂. By such processes, billions of tons of CO₂ are emitted into the atmosphere per year.

Now, it is an object to provide a method that is capable of generating other energy carriers, for example as fuels or combustibles. The provision of the energy carriers should be preferably without emission of CO₂.

### SUMMARY OF THE INVENTION

According to the invention, a method is proposed for providing storable and transportable energy carriers. In one step, transformation of silicon-dioxide-containing or metal-oxide-containing starting material (herein also termed oxygen-containing starting material) to silicon or a metal occurs in a reduction process, wherein the primary energy for this reduction process is provided from a renewable energy source. A portion of the reduction products of the reduction process is then applied in a process for generating methanol, wherein, a synthesis gas composed of carbon monoxide and oxygen is used.

The industrial extraction of metals from their oxides by carbo-thermal and electrolytical processes requires high temperatures and produces large amounts of green-house gases (e.g. CO₂). It is an advantage of the invention that the required energy input originates wholly or partially from renewable energy sources (e.g. solar energy) and that thereby no or hardly any green-house gases are emitted.

Further preferable embodiments can be taken from the description, the figures and the claims.

### BRIEF DESCRIPTION OF THE DRAWING

In the figures, different aspects of the invention are represented schematically, wherein:

FIG. 1: shows a schematic illustrating the basic steps of a first method according to the invention;

FIG. 2: shows a schematic illustrating the basic steps of a second method according to the invention;

FIG. 3: shows a schematic illustrating the basic steps of a third method according to the invention;

FIG. 4: shows a schematic illustrating the basic steps of a fourth method according to the invention;

FIG. 5: shows a schematic illustrating the basic steps of a fifth method according to the invention;

FIG. 6: shows a schematic illustrating partial steps of a further method according to the invention; and

FIG. 7: shows a schematic illustrating partial steps of a further method according to the invention.

### DETAILED DESCRIPTION

The method according to the invention is based on a novel concept, which provides so-called reaction products under application of existing starting materials, which reaction products can be utilized either directly as energy carriers, or which can then further processed in intermediate steps be utilized as energy carriers.

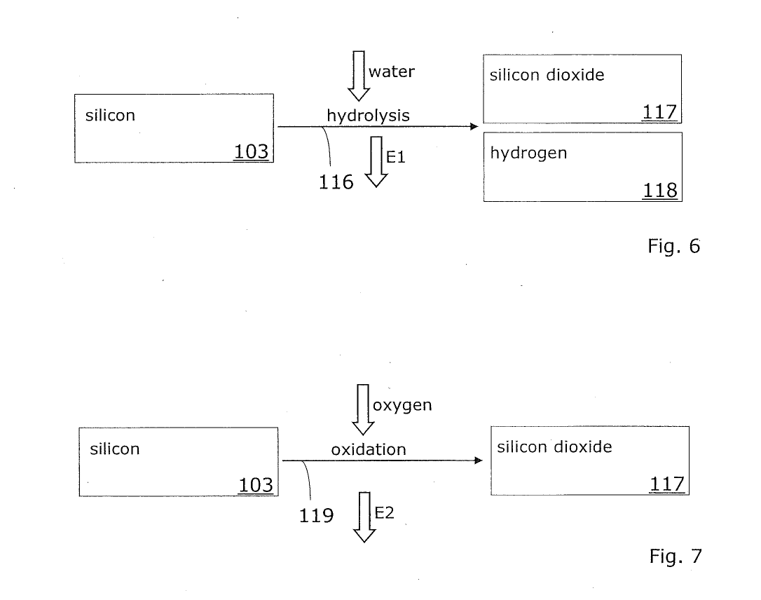

The term “energy carrier” is used herein to designate compounds, which can be used as a fuel or combustible directly (such as, e.g., methanol 104 or hydrogen 118) and also compounds (such as, e.g., silicon 103 or elementary metals), which have an energy content or an elevated energy level and which can be converted in further steps with delivery of energy (refer to the energy E1 and E2 in FIGS. 6 and 7) and/or with delivery of a further energy carrier (such as, e.g., hydrogen 118).

The transportability of an energy carrier is characterized herein by the chemical reaction potential. For a safe transportability of the energy carrier, this reaction potential should be as low as possible. In the case of silicon 103 as an energy carrier, specific framework conditions concerning the storage and transport should be observed, so as to avoid initiating an undesired or uncontrolled reaction (oxidation) of the silicon or the metal. The silicon 103 or the metal should preferably be stored and transported in a dry state. In addition, the silicon 103 or the metal should not be heated, because otherwise the probability of a reaction with water vapor from the ambient air or with oxygen increases.

Investigations have shown that, e.g., silicon up to approximately 300° C. has only a little tendency of reacting with water or oxygen. It is ideal to store and transport the silicon 103 or the metal together with a water-getter (i.e. a compound that is hydrophilic attracting water) and/or an oxygen-getter (i.e. a compound attracting oxygen).

The term silicon-dioxide-containing or metal-oxide-containing starting material 101 is used herein to designate compounds which contain a large portion of silicon dioxide (SiO₂) or a large portion of at least one metal oxide (e.g. bauxite).

Sand and/or shale (SiO₂+[CO₃]²) are particularly suitable. Sand is a naturally occurring uncompacted sedimentary rock and occurs everywhere on the surface of the Earth in more or less large concentrations. A majority of the occurrences of sand consists of quartz (silicon dioxide; SiO₂).

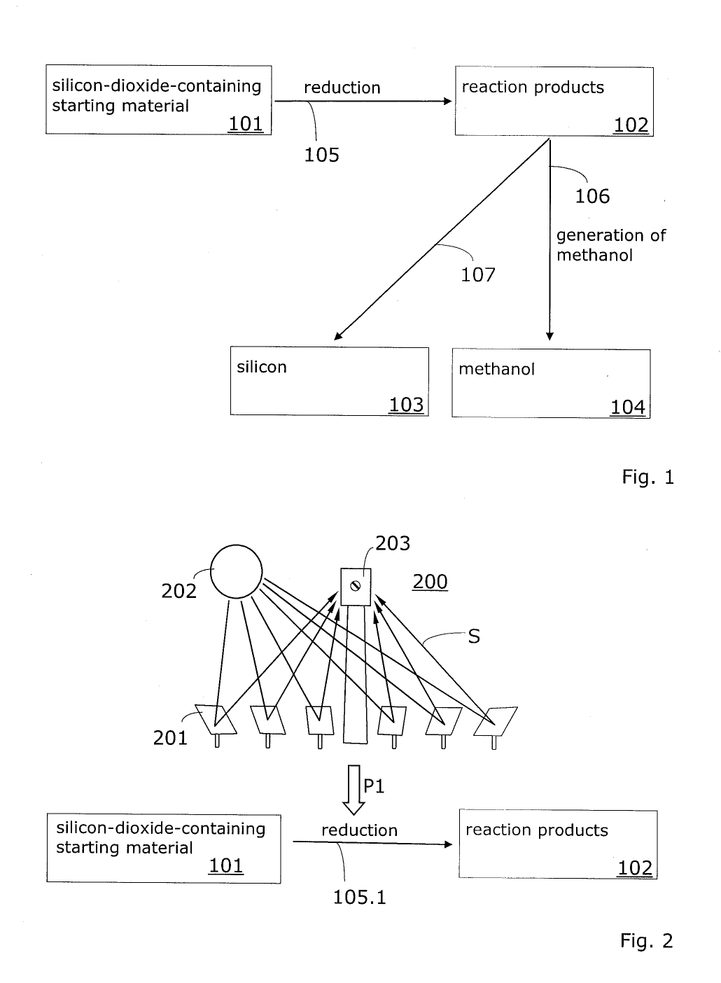

In FIG. 1, the basic steps of a first method according to the invention for providing storable and transportable energy carriers 103, 104 are shown. In this method, silicon 103, as a first storable and transportable energy carrier, and methanol 104, as a second storable and transportable energy carrier, are provided. The method comprises at least the following steps.

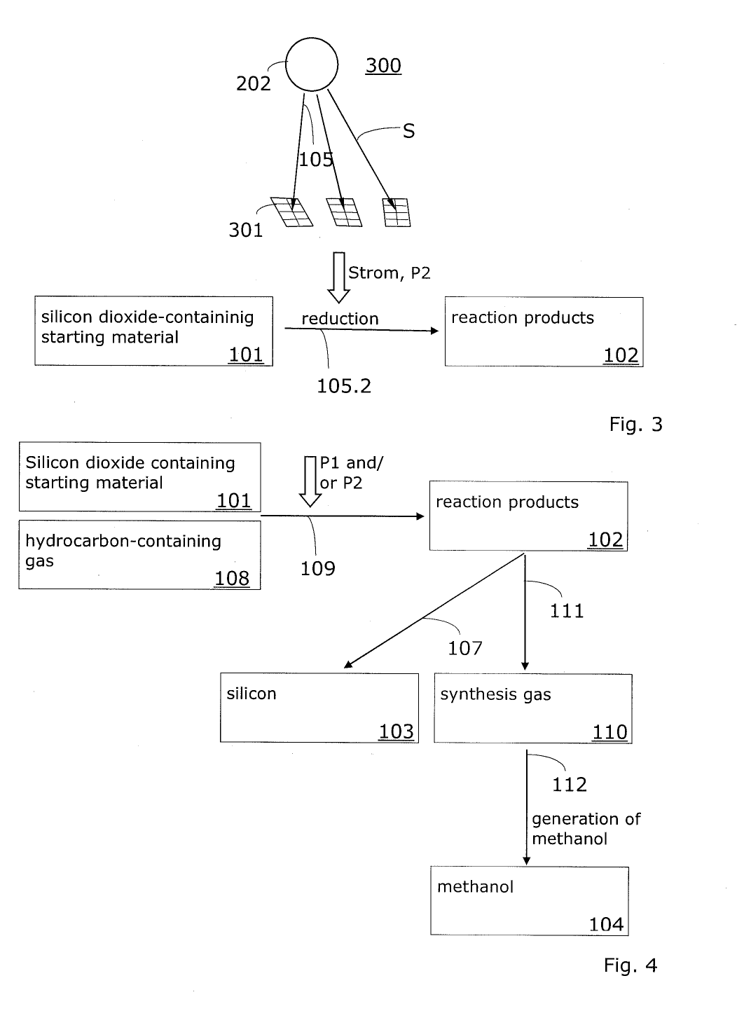

By a transformation, a silicon-dioxide-containing starting material 101 is converted into elementary silicon 103 by means of an endothermal reduction process 105. The elementary silicon 103 is called herein silicon for reasons of simplicity. According to the invention, the required primary energy (refer to primary energy P1 in FIG. 2 or primary energy P2 in FIG. 3) for this reduction process 105 is provided from a renewable energy source. In a subsequent (downstream) step, at least a portion of the reaction products 102 of the reduction process 105 is utilized in a process 106 for generating methanol. In this process 106 for generating methanol, a synthesis gas 110 composed of carbonic oxide gas (CO) and hydrogen (H₂) or composed of carbonic acid gas (CO₂) and hydrogen (H₂) comes to application. In FIG. 1 it is further indicated schematically that the silicon 103 can be taken from the method as a first energy carrier. The extraction of the silicon 103 is indicated in FIG. 1 as a method step 107. The silicon 103 can for example be stored or transported away. The more silicon 103 that is extracted, the less silicon 103 can be utilized as an energy supplier in the process 106 for generating methanol.

Basic details concerning solar-thermal processes can be taken from the book of Steinfeld A., Palumbo R., “Solar Thermochemical Process Technology”, Encyclopedia of Physical Science and Technology, Academic Press, ISBN 0-12-227410-5, vol. 15, pp. 237-256, 2001.

The transformation 105 is preferably a thermo-chemical transformation 105.1 (under application of heat energy), as indicated schematically in FIG. 2, or an electro-chemical transformation 105.2 (under application of electric current) as indicated schematically in FIG. 3. The process 105.1 in FIG. 2 is also termed thermal dissociation, and this process can be described by the following equation (1) (wherein M designates herein a metal or silicon and MₓO_{y} a metal oxide or silicon dioxide):

MₓO_{y}→xM+0.5yO₂(g) (1)

In the thermo-chemical transformation 105.1 of FIG. 2, the primary energy P1 for the transformation is provided by sunlight S. For the thermo-chemical transformation 105.1, a solar heat plant 200 is utilized, as indicated schematically in FIG. 2. The solar heat plant 200 comprises a plurality of rotatable heliostats 201 which can preferably be tracked with the movement of the sun 202. The heliostats 201 reflect the sunlight S in the direction of a solar tower 203. In the focal point of the sunlight S, extremely high temperatures are achieved. In FIG. 2 it is indicated schematically by a block arrow P1 that the heat energy provided by the solar heat plant 200 comes to application so as to actuate and energize the endothermal reduction process 105.1. Depending on the embodiment, the solar energy can act directly on the silicon-dioxide-containing starting material 101, or a liquid transfer medium (typically air, water, synthetic oil, helium, sodium, molten salt) can be utilized as an agent for the dissemination/transfer of the energy P1. A catalyst is preferably utilized to facilitate or accelerate the reduction reaction in the reduction process 105.1.

In the electro-chemical transformation 105.2 according to FIG. 3, the primary energy P2 for the transformation is delivered by electric current that is produced from sunlight S. For the electro-chemical transformation 105.2, a solar energy plant 300 is utilized, as indicated schematically in FIG. 3. The solar energy plant 300 comprises a plurality of (rotatable) solar modules 301 which can preferably be tracked with the movement of the sun 202. The solar modules 301 convert the sunlight S to electric current. The electro-chemical transformation 105.2 can for example be performed by utilizing silicon dioxide as an electrode. A metal is utilized as a second electrode. As an electrolyte, for example calcium chloride (CaCl₂) or another electrolyte, preferably a chloride-containing electrolyte, is utilized. Also NH₄Cl, for example, is suitable. This electro-chemical transformation process 105.2 works particularly well when using a porous electrode made of silicon dioxide, which can for example be sintered from silicon dioxide. Details concerning this process can be taken from the following publications:
Nature materials 2003 June; 2 (6): 397-401, Nohira T., Yasuda K., Ito Y., Publisher: Nature Pub Group;
“New silicon production method with no carbon reductant”, George Zheng Chen; D. J. Fray, T. W. Farthing, Tom W. (2000);
“Direct electrochemical reduction of titanium dioxide to titanium in molten calcium chloride”, George Zhen Chen, D. J. Fray, T. W. Farthing, Nature 407 (6802): 361-364. doi: 10.1038/35030069;
“Effect of electrolysis potential on reduction of solid silicon dioxide in molten CaCl₂”, YASUDA Kouji; NOHIRA Toshiyuki; ITO Yasuhiko; The Journal of physics and chemistry of solids, ISSN 0022-3697, International IUPAC Conference on High Temperature Materials Chemistry No. 11, Tokyo, Japan (19 May 2003), 2005, vol. 66, no. 2-4 (491 p.);
U.S. Pat. No. 6,540,902 B1;
WO 2006 092615 A1.

Preferably, the reduction process 105.1 is performed at a temperature of approximately 1900° Kelvin (=1630° C.), in order to reduce the silicon dioxide to silicon (Si). When a catalyst is utilized, the reduction process temperature is somewhat lower. In the electro-chemical transformation 105.2, significantly lower temperatures (preferably less than 500° C.) are required.

An electro-chemical transformation 105.2 is particularly suitable because, beside the provision of heat energy, it is also supported by electric current so as to be able to set the temperature required for the endothermal reduction lower. Additionally or alternatively to the input of electric current, it is however also possible to utilize a reduction agent and/or a catalyst. It is a disadvantage that here, though, depending on the process control and the reduction agent, CO₂ can be generated according to circumstances.

Preferably, the reduction processes 105.1, 105.2 are performed in an oxygen-poor or an oxygen-free environment, because otherwise the elementary silicon 103 occurring in the reduction would (re-)oxidize immediately. In addition, oxygen together with silicon form a layer of silicon dioxide on the melt, which may hinder the reduction process.

Therefore, a process is ideal in which the starting material (e.g. sand) is supplied via a drop distance so as to offer a surface that is as large as possible for the reduction process 105, 105.1, 105.2. The starting material (e.g. sand) may however also be vortexed, stirred, blown up or foamed so as to offer a large surface for the reduction process 105, 105.1, 105.2.

A further method according to the invention is shown in FIG. 4. A scheme is illustrated which represents the basic steps of a fourth method according to the invention. Here, the reduction process 109 is performed under supply of a hydrocarbon-containing gas 108. Preferably, methane (CH₄), biogas or natural gas (natural gas: NG) is utilized as the hydrocarbon-containing gas 108. In the reduction process 109 (also called methano-thermal reduction), the following reaction products 102 are generated:

silicon 103,

carbonic oxide gas and/or carbonic acid gas and

hydrogen.

This reduction process 109 can be described by the following equation:

MₓO_{y}+yCH₄→xM+y(2H₂+CO) (2)

It is important that the hydrocarbon-containing gas 108 is dosed in the process to avoid silicon carbide (SiC) being formed instead of silicon as a consequence of a surplus of carbon (C).

The term biogas is used herein to denominate gases which may be generated, e.g., by a fermentation process under exclusion of air. Examples of biogases are the gases from sewage treatment plants, from useful animal husbandry, but also gases that are provided by facilities which convert biomass. Here, preferably, only biogases come to application, which originate from renewable sources and which are not in concurrency with the cultivation of food products. The methane can for example, be produced in a pyrolysis process, wherein the pyrolysis process is powered by biomass.

In this fourth method according to the invention, the hydrocarbon-containing gas 108 is applied on one hand to serve as a reduction agent for the reduction of the silicon dioxide or of another starting material. On the other hand, the hydrocarbon-containing gas 108 serves as a “starting material” for the provision of the synthesis gas composed of carbonic oxide gas and/or carbonic acid gas and hydrogen. The following reaction (3) takes place, e.g. according to FIG. 4:

SiO₂+CH₄(g)→Si+2CO+4H₂(g) (3)

The reaction equation (3) reflects a method according to FIG. 4, in which methane is utilized as the hydrocarbon-containing gas 108. The “breakdown” of CH₄ into the synthesis gas 110 requires a supply of energy. The synthesis gas 110 comprises a mixture of H₂ and CO, which mixture is particularly suitable for synthesizing methanol therefrom. Here, the corresponding energy [Δ_{R}H approx. 160 kJ/mol] is delivered from renewable energy sources (e.g., one of the plants 200 or 300]. That is, the CH₄ is not utilized as an energy supplier for this step 109. In a reaction according to equation (3), the reduction of a metal (here silicon dioxide) is combined with a reformation of the methane gas. In order to perform this reaction, the energy must be supplied from the outside. In FIG. 4, the energy supply is indicated by a block arrow referenced with P1 and/or P2. That is, the energy can, e.g., originate from a solar heat plant 200 and/or from a solar energy plant 300. The energy may however also originate from other sources (e.g. hydropower, wind power or fossil forms of energy).

Instead of the reaction (3), the following carbo-thermal reduction can also be carried out:

MₓO_{y}+0.5yC→xM+0.5yCO₂ (3.1)

In the method according to FIG. 4, the silicon dioxide of the silicon-dioxide-containing material 101 functions as a donor of oxygen.

The synthesis gas 110 (here, e.g., 2 CO+4H₂ (g)) is further converted here to methanol 104 in a method 112 for generating methanol.

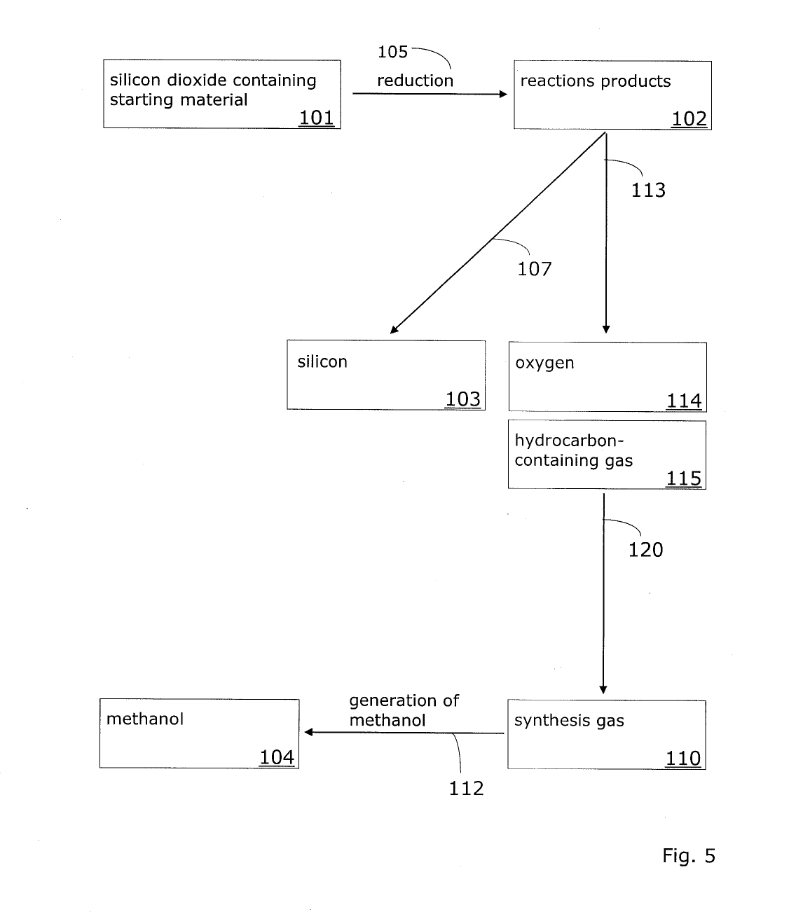

A further method according to the invention is shown in FIG. 5. A scheme is illustrated, which corresponds in part to the method of FIG. 1. However, further method steps are appended here with respect to the method of FIG. 1. Here, in the reduction process 105, silicon 103 and oxygen 114 are generated as reaction products 102. Here, the oxygen 114 is converted under supply of a hydrocarbon-containing gas 115 to a synthesis gas 110 composed of carbonic oxide gas (and/or carbonic acid gas) and hydrogen. The method step 120 concerns a gas oxidation process. The gas oxidation process is slightly exothermal. Preferably, methane (CH₄), biogas, or natural gas (NG) is utilized as the hydrocarbon-containing gas 115. Here, the synthesis gas 110 is then further converted to methanol 104 in a process 112 for generating methanol.

In relation with FIGS. 6 and 7 it is described using examples, how silicon 103 can be utilized as an energy carrier. The reduced silicon 103 is an energy-rich compound. This silicon has a tendency to (re)oxidize again with water in liquid or vapour form to silicon dioxide 117, as schematically shown in FIG. 6. This process is also a called “wet-oxidation” and is known from the area of semiconductor technology as a surface process, but is however not used in the area of energy transport or energy generation. In the so-called hydrolysis 116 of the silicon 103, energy E1 is liberated, because an exothermal reaction is concerned. The reaction takes place according to the simplified equation:

Si+2H₂→SiO₂+2H₂ (4)

In addition to the silicon dioxide 117, hydrogen 118 is generated, which can for example be utilized as an energy carrier or fuel. Preferably, the hydrolysis 116 takes place at elevated temperatures. Temperatures, which are significantly above 100° C., are preferred. In the temperature range between 100° C. and 300° C., a conversion in usable quantities is achieved in cases when the silicon has a very fine-grained or a powdery consistency and is brought in contact with water vapor and stirred. Since otherwise silicon up to approximately 300° C. has only a very low tendency to react with water, the hydrolysis 116 is preferably carried out at temperatures in a temperature range between 300° C. and 600° C.

According to the invention, in a method according to FIG. 6, the silicon 103 is introduced into a reaction area and mixed with water in liquid or vapor form. In addition, according to the invention, care is taken that the silicon 103 has a minimum temperature. To this end, the silicon 103 is either heated (e.g. using heating means or by means of heat-generating or heat-delivering additives) or the silicon 103 is at a corresponding temperature level already when it is introduced.

Under these framework conditions, hydrogen is then liberated in the reaction area in gaseous form. The hydrogen is extracted from the reaction area.

In the following, a numerical example is given for a method according to FIG. 1 in combination with FIG. 6 or according to FIG. 5 in combination with FIG. 6:

1 Mol (=60.1 g) SiO₂ yields 1 Mol (=28 g) Si. 1 Mol (=28 g) Si in turn yields 1 Mol (=451 g) H₂. That is, 2.15 kg SiO₂ form 1 kg Si, and from this 1 kg Si, 1.6 m³ H₂ is generated.

The silicon 103 also has the tendency to oxidize again with oxygen to silicon dioxide 117, as represented in FIG. 7. An energy E2 is liberated, because an exothermal reaction is concerned. Preferably, the oxidation 119 takes place in a temperature range between 500° C. and 1200° C. Temperatures are preferred which are above 1000° C. The corresponding temperature can e.g. be provided using a solar heat plant 200 or a solar energy plant 300.

The method according to FIG. 7 can, for example, be carried out in an oxidation oven. Preferably, a thermal oxidation is performed in the oxidation oven, wherein the energy for initiating (energizing the oxidation originates from renewable energy sources (preferably from solar energy).

The oxidation of the silicon 103 should preferably be carried out with dry oxygen so as to exclude a simultaneous concurrent hydrolysis process.

The method according to FIG. 7 can, for example, also be carried out in a plasma oxidation oven. Here, only temperatures in a temperature range between 300° C. and 600° C. are required, since a portion of the required energy is provided by the plasma.

A further aspect of the invention is the conversion of CO₂ to CO. A direct conversion requires temperatures in the range of far beyond 2000° C. and is therefore not economical depending on the circumstances. However, there is the known approach to conduct the conversion via the so-called water gas-shift-reaction, which takes place according to the following equation (5):

CO₂+H₂(g) [symbol] CO(g)+H₂O (5)

The ΔH in this reaction (5) is close to +41.19 kJ. At about 830° C., there is a 1:1 mixture of CO and CO₂ (i.e. at approx. 830° C. the equilibrium constant K is close to one, i.e. K=1). From this mixture, CO can easily be separated from CO₂. The CO₂ can then, for example, be supplied back to this reaction (5). The required temperature can, for example, be generated by a mirror arrangement (e.g. a parabolic mirror). A synthesis gas can then be produced from the CO, and methanol can be produced from the synthesis gas.

The conversion of CO₂ to CO may, however, also take place according to the following inventive principal:

MₓO_{y}→xM+0.5yCO₂ (6.1)

xM+yCO₂→MₓO_{y}+yCO (6.2)

The CO can be utilized as a combustible or can be converted together with hydrogen to methanol, as described above. Thus it is possible to reduce a silicon-dioxide-containing or a metal-oxide-containing starting material 101 in a reduction process to the corresponding metal, for example in sun-rich regions or at sites where other renewable energy forms are available. The metal can be utilized at another site (e.g. in the vicinity of an industrial facility or a power plant) so as to convert the CO₂ that is generated into CO.

A reduction process according to equation (6.3) is particularly preferred, wherein the water is utilized together with the CO₂ so as to generate a synthesis gas.

2xM+yH₂O+yCO₂→2MₓO_{y}+yCO+yH₂ (6.3)

In a mixture composed of CO and CO₂, the CO₂ can simply be separated into water, methanol or other alcohols of CO by dissolution, because the CO does not dissolve or dissolves hardly at all.

In the hydrolysis 116 of the silicon 103 or the metal, hydrogen is generated as described. This hydrogen can, as shown in the equation (5), be converted together with CO₂ (for example CO₂ from flue gases) to CO. Then methanol can be produced from CO plus a portion of hydrogen (synthesis gas).

The generation of methanol can be performed according to one of the methods which are known and utilized at large scale. A method is preferred in which a catalyst (e.g. a CuO—ZnO—Cr₂O₃ or a Cu—Zn—Al₂O₃ catalyst) is utilized.

The invention has the advantage that in the reduction of the silicon dioxide or of one of the other metal oxides, no CO₂ or less CO₂ is disseminated. The required energy is provided from renewable energy sources, preferably from solar power plants 200 or 300.

The elementary silicon 103 is applied preferably in a powdery form or in a granular or grainy form so as to offer a preferably large surface in the oxidation (refer to step 119 in FIG. 7) or in the hydrolysis (refer to step 116 in FIG. 6).

Silicon plays an essential role for electronic components, such as solar cells and semiconductor chips, as well as for the generation of polysiloxanes. The elementary silicon 103 can thus also be further processed or graded up in a corresponding process.

The processes according to the invention are characterized by the fact, that they do not necessarily concern circulation processes, in which the products (e.g. the silicon dioxide or a metal oxide) is lead back to the beginning of the process for then being reduced again (e.g., to silicon or a metal). Due to the fact that silicon dioxide is a cheap starting material, the circulation can be designed openly. In this case the silicon dioxide which is generated at the end, or the metal oxide generated, are extracted from the process so as to be utilized, for example, for the manufacturing of glass.

In order to facilitate or accelerate the reduction reaction of the different reduction processes 105, 105.1, 105.2, 109, a catalyst and/or a reduction agent is preferably utilized. Beside the carbon or hydrocarbon for a so-called carbo-thermal reduction, metals also may serve as a reduction agent. Here, for example, it is possible to utilize magnesium (Mg) or zinc (Zn). The magnesium (Mg) can be produced using an electro-thermal reduction (analogously to FIG. 2) from MgO and the zinc from ZnO.

The thermal dissociation according to equation (1) can preferably be linked with an oxidation process for the generation of methanol. In this oxidation process, a hydrocarbon (e.g. methane gas) is brought together with the oxygen from the reaction of equation (1) and converted to methanol. The methanol can be generated by means of a direct oxidation or through a partial oxidation or through a reforming. Details in this respect can be taken from the parallel application PCT/EP2009/061707), which has been filed on 9 Sep. 2009.

1. The method for providing storable and transportable energy carriers, the method comprising the following steps:
transformation of a silicon-dioxide-containing or a metal-oxide-containing starting material to silicon or a metal reaction products in a reduction process wherein primary energy for this reduction process is provided from a renewable energy source,
application of a portion of the reaction products of the reduction process in a process for generating methanol, wherein in the process for generating methanol involves a synthesis gas composed of carbon monoxide and/or hydrogen, comes to application.

2. A method according to claim 1, wherein the transformation is a thermo-chemical or an electro-chemical transformation.

3. A method according to claim 2, wherein the primary energy for the transformation is delivered by sunlight through a solar heat plant or a solar.

4. A method according to claim 1, wherein the reduction process is performed at a temperature of approximately 1900 Kelvin (=1630° C.).

5. A method according to claim 1, wherein the reduction process is performed in an oxygen-poor or oxygen-free environment.

6. A method according to claim 1, wherein the reduction process is performed under supply of a hydrocarbon-containing gas selected from the group consisting of methane, biogas and natural gas (NG), wherein the following reaction products of the reduction process are achieved:
silicon or elementary metal,
carbon monoxide and/or carbon dioxide and
hydrogen.

7. A method according to claim 6, wherein the silicon is provided as a first storable and transportable energy carrier and, in the process for generating methanol, methanol is provided as a second storable and transportable energy carrier from the carbon monoxide and/or the carbon dioxide and/or the hydrogen.

8. A method according to claim 6, wherein energy for converting the hydrocarbon-containing gas is provided from solar energy.

9. A method according to claim 1, wherein the following reaction products of the reduction process are provided:
silicon; and
oxygen.

10. A method according to claim 8, wherein the silicon is provided as a first storable and transportable energy carrier and in a gas oxidation process oxygen is converted to the synthesis gas composed of carbon monoxide and/or carbon dioxide and hydrogen using a supply of a hydrocarbon-containing gas selected from the group consisting of methane, biogas or natural gas (NG).

11. A method according to claim 10, wherein methanol is provided as a second storable and transportable energy carrier from the carbon monoxide and/or carbon dioxide and the hydrogen.

12. A method according to claim 1, wherein silicon is provided as a first storable and transportable energy carrier, wherein in a further step liquid water or water vapor is brought in contact with said silicon so as produce hydrogen, silicon dioxide and a first amount of energy in a hydrolysis reaction.

13. A method according to claim 1, wherein silicon is provided as a first storable and transportable energy carrier, wherein in a further step oxygen is brought in contact with the silicon so as to produce silicon dioxide and a second amount of energy in an oxidation reaction.

14. A method according to claim 1, wherein hydrogen is produced in a hydrolysis step wherein in a further step, hydrogen reacts with carbon dioxide to form carbon monoxide and water.

15. A method according to claim 14, wherein in a further step, a portion of the hydrogen together with the carbon monoxide is converted to methanol.

16. A method according to claim 14, wherein carbon dioxide is extracted from a combustion process.

17. A method according to claim 1, wherein the method is performed in open circulation and comprises the following steps:
performing the reduction process at a first site so as to provide said reaction products,
transporting a portion of the reaction products to a second site,
reacting said portion of said reaction products at the second site.
